(19) United States
(12) Patent Application Publication
(10) Pub. No.: US 2026/0207178 A1
(43) Pub. Date: Jul. 23, 2026

(54) ULTRASONIC IMAGING APPARATUS AND ACOUSTIC CHARACTERISTICS CORRECTION METHOD THEREOF

(71) Applicant: SAMSUNG MEDISON CO., LTD., Hongcheon (KR)

(72) Inventors: Seoksoon NOH, Hongcheon (KR); Sungho Kim, Hongcheon (KR)

(21) Appl. No.: 19/308,196

(22) Filed: Aug. 23, 2025

(30) Foreign Application Priority Data
Jan. 23, 2025 (KR) 10-2025-0010097

(51) Int. Cl. A61B 8/00

(52) U.S. Cl. CPC A61B 8/58; A61B 8/4444

(57) ABSTRACT

Disclosed is an acoustic characteristics correction method of an ultrasonic imaging apparatus, which includes a main body including a transmission channel configured to generate a transmission signal based on a synchronization signal, and a probe including a plurality of elements configured to generate an ultrasonic signal based on the transmission signal, including obtaining a pre-correction parameter about the probe based on connection of the main body and the probe, determining a transmission parameter based on at least one of an object or a diagnostic subject, correcting at least one of a preset transmission delay time or a preset transmission intensity corresponding to the transmission parameter based on the pre-correction parameter, controlling the transmission channel to generate a transmission signal based on at least one of the corrected transmission delay time or the corrected transmission intensity, and generating and outputting an ultrasonic signal in the element connected to the transmission channel based on the generated transmission signal.

### CROSS-REFERENCE TO RELATED APPLICATION(S)

This application is based on and claims priority under 35 U.S.C. § 119 to Korean Patent Application No. 10-2025-0010097, filed on Jan. 23, 2025, in the Korean Intellectual Property Office, the disclosure of which is incorporated by reference herein in its entirety.

### BACKGROUND

### 1. Field

The disclosure relates to an ultrasonic imaging apparatus and an acoustic characteristics correction method thereof.

### 2. Description of the Related Art

Recently, in a medical field, various medical imaging apparatuses have been widely used to image and obtain information about biological tissues of a human body for the purpose of early diagnosis of various diseases or surgery. Representative examples of such medical imaging apparatuses may include ultrasonic imaging apparatuses, computed tomography (CT) apparatuses, and magnetic resonance imaging (MRI) apparatuses.

An ultrasonic imaging apparatus is a device that emits an ultrasonic signal generated from an element, which is an ultrasonic transmission/reception unit constituting a transducer of a probe, to an object, and non-invasively obtains at least one image of a region inside the object (e.g., soft tissue or blood flow) by receiving information from the signal reflected from the object. In particular, an ultrasonic imaging apparatus is used for medical purposes such as observing the inside of an object, detecting foreign substances, and measuring injury. Such an ultrasonic imaging apparatus is widely used together with other diagnostic apparatuses because the ultrasonic imaging apparatus has higher stability than an imaging apparatus using an X-ray, may display images in real time, and is safe because there is no radiation exposure.

Conventional developers have to invest more time in development because they need to find optimal image conditions and set the latency to match a probe to a transducer, which causes inconvenience.

Conventional developers have to invest more time in development because latency of a probe needs to be set to match a transducer to find optimal imaging conditions.

In using a conventional ultrasonic diagnostic apparatus in which a probe is connected to a main body, acoustic characteristics deviations occur due to unevenness in signal quality caused by dispersion in ultrasonic probe production process, such as a design and manufacturing process of components inside the apparatus (e.g., main body, probe, PCB of cables, etc.).

Conventionally, a method of correcting such acoustic characteristics deviations through a delay time of a transmission signal based on information about a distance between a focus position and a probe element (i.e., In Air TOF) has been used. However, deviations in unique acoustic characteristics of components inside the apparatus that occur in the design or manufacturing process and deviations in acoustic characteristics that occur in a signal processing process are not reflected, thereby deteriorating the quality of an ultrasonic image.

### SUMMARY

It is an aspect of the disclosure to provide an ultrasonic imaging apparatus and an acoustic characteristics correction method thereof capable of obtaining an image of improved quality by correcting minute acoustic characteristics deviations.

Additional aspects of the disclosure will be set forth in part in the description which follows and, in part, will be obvious from the description, or may be learned by practice of the disclosure.

An aspect of the disclosure provides an acoustic characteristics correction method of an ultrasonic imaging apparatus, which includes a main body including a transmission channel configured to generate a transmission signal based on a synchronization signal, and a probe including a plurality of elements configured to generate an ultrasonic signal based on the transmission signal, including obtaining a pre-correction parameter about the probe based on connection of the main body and the probe, determining a transmission parameter based on at least one of an object or a diagnostic subject, correcting at least one of a preset transmission delay time or a preset transmission intensity corresponding to the transmission parameter based on the pre-correction parameter, controlling the transmission channel to generate a transmission signal based on at least one of the corrected transmission delay time or the corrected transmission intensity, and generating and outputting an ultrasonic signal in the element connected to the transmission channel based on the generated transmission signal.

Another aspect of the disclosure provides an ultrasonic imaging apparatus comprising a main body comprising: a transmission module comprising a transmission channel configured to generate a transmission signal based on a synchronization signal, a main body communication module configured to perform communication with an external device including a probe, and a processor electrically connected to the transmission module and the main body communication module; and a probe comprising a plurality of elements configured to generate an ultrasonic signal based on the transmission signal, wherein the processor may be configured to obtain a pre-correction parameter about the probe based on connection of the main body and the probe, determine a transmission parameter based on at least one of an object or a diagnostic subject, correct at least one of a preset transmission delay time or a preset transmission intensity corresponding to the transmission parameter based on the pre-correction parameter, and control the transmission channel to generate a transmission signal based on at least one of the corrected transmission delay time or the corrected transmission intensity.

### BRIEF DESCRIPTION OF THE DRAWINGS

These and/or other aspects of the disclosure will become apparent and more readily appreciated from the following description of the embodiments, taken in conjunction with the accompanying drawings of which:

FIG. 1 illustrates a control block diagram of an ultrasonic imaging system 100 in a case in which a probe 20 is a wired probe or a hybrid probe;

FIG. 2 illustrates a control block diagram of the ultrasonic imaging system 100 in a case in which the probe 20 is a wireless probe or a hybrid probe;

FIGS. 3 to 6 are views illustrating the ultrasonic imaging system 100 according to an embodiment;

FIG. 7 is a view illustrating a portion of the probe 20 according to an embodiment;

FIG. 8 is an enlarged view of a region A of the probe 20 illustrated in FIG. 7 according to an embodiment;

FIG. 9 is a diagram for explaining a method of obtaining a third pre-correction sub-parameter for correcting an acoustic characteristics deviation due to unique characteristics of a type of the probe 20 according to an embodiment;

FIG. 10 is a diagram for explaining a post-correction parameter according to an embodiment;

FIG. 11 is a diagram for explaining generating and/or correcting a transmission signal according to an embodiment;

FIG. 12 is a flowchart of a method of correcting an acoustic characteristics deviation using a pre-correction parameter according to an embodiment; and

FIG. 13 is a flowchart of a method of correcting an acoustic characteristics deviation using a post-correction parameter according to an embodiment.

### DETAILED DESCRIPTION

This disclosure will explain the principles and disclose embodiments of the disclosure to clarify the scope of the claims of the disclosure and enable those skilled in the art to which the embodiments of the disclosure belong to practice the embodiments. The embodiments of the disclosure may be implemented in various forms.

Throughout the specification, like reference numbers refer to like elements throughout this specification. This specification does not describe all components of the embodiments, and general contents in the technical field to which the disclosure belongs or overlapping contents between the embodiments will not be described. The “module” or “unit” used in the specification may be implemented as one or a combination of two or more of software, hardware, or firmware, and according to embodiments, a plurality of “module” or “unit” may be implemented as a single element, or a single “module” or “unit” may include a plurality of elements.

The singular form of a noun corresponding to an item may include a single item or a plurality of items, unless the relevant context clearly indicates otherwise.

In this disclosure, each of phrases such as “A or B,” “at least one of A and B,” “at least one of A or B,” “A, B or C,” “at least one of A, B and C,” and “at least one of A, B, or C” may include any one of the items listed together in the corresponding one of the phrases, or all possible combinations thereof.

The term “and/or” includes any combination of a plurality of related components or any one of a plurality of related components.

The terms such as “first,” “second,” “primary,” and “secondary” may simply be used to distinguish a given component from other corresponding components, and do not limit the corresponding components in any other respect (e.g., importance or order).

The terms “front surface,” “rear surface,” “upper surface,” “lower surface,” “side surface,” “left side,” “right side,” “upper portion,” “lower portion,” and the like used in the disclosure are defined with reference to the drawings, and the shape and position of each component are not limited by these terms.

The terms “comprises,” “has,” and the like are intended to indicate that there are features, numbers, steps, operations, components, parts, or combinations thereof described in the disclosure, and do not exclude the presence or addition of one or more other features, numbers, steps, operations, components, parts, or combinations thereof.

When any component is referred to as being “connected,” “coupled,” “supported,” or “in contact” with another component, this includes a case in which the components are indirectly connected, coupled, supported, or in contact with each other through a third component as well as directly connected, coupled, supported, or in contact with each other.

When any component is referred to as being located “on” or “over” another component, this includes not only a case in which any component is in contact with another component but also a case in which another component is present between the two components.

Hereinafter, an ultrasonic apparatus according to various embodiments will be described in detail with reference to the accompanying drawings. When described with reference to the accompanying drawings, similar reference numbers may be assigned to identical or corresponding components and redundant description thereof may be omitted.

In this disclosure, an image may include a medical image acquired by a medical imaging apparatus such as a magnetic resonance imaging (MRI) apparatus, a computed tomography (CT) apparatus, an ultrasonic imaging apparatus, and an X-ray imaging apparatus.

In this disclosure, an ‘object’, which is subject to photography, may include a person, animal, or part thereof. For example, the object may include a part of a human body (an organ, etc.) or a phantom.

In this disclosure, an ‘ultrasonic image’ refers to an image of an object that has been generated or processed based on an ultrasonic signal transmitted to and reflected from the object.

Hereinafter, embodiments of the disclosure will be described in detail with reference to the drawings.

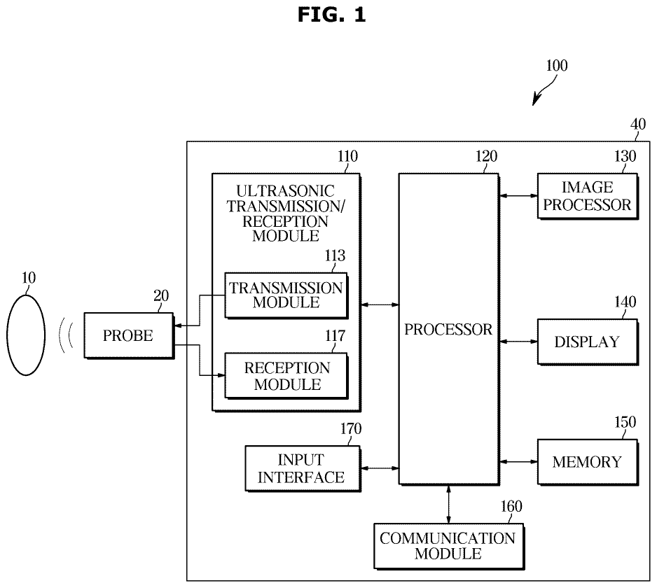

Referring to FIGS. 1A and 1B, an ultrasonic imaging system 100 may include a probe 20 and an ultrasonic imaging apparatus 40.

The ultrasonic imaging apparatus 40 may be implemented not only in a cart type but also in a portable type. A portable ultrasonic imaging apparatus may include, for example, a smart phone, a laptop computer, a personal digital assistant (PDA), a tablet PC, etc., which include a probe and an application, but is not limited thereto.

The probe 20 may include a wired probe connected to the ultrasonic imaging apparatus 40 by wire to communicate with the ultrasonic imaging apparatus 40 by wire, a wireless probe wirelessly connected to the ultrasonic imaging apparatus 40 to communicate wirelessly with the ultrasonic imaging apparatus 40, and/or a hybrid probe connected to the ultrasonic imaging apparatus 40 by wire or wirelessly to communicate with the ultrasonic imaging apparatus 40 by wire or wirelessly.

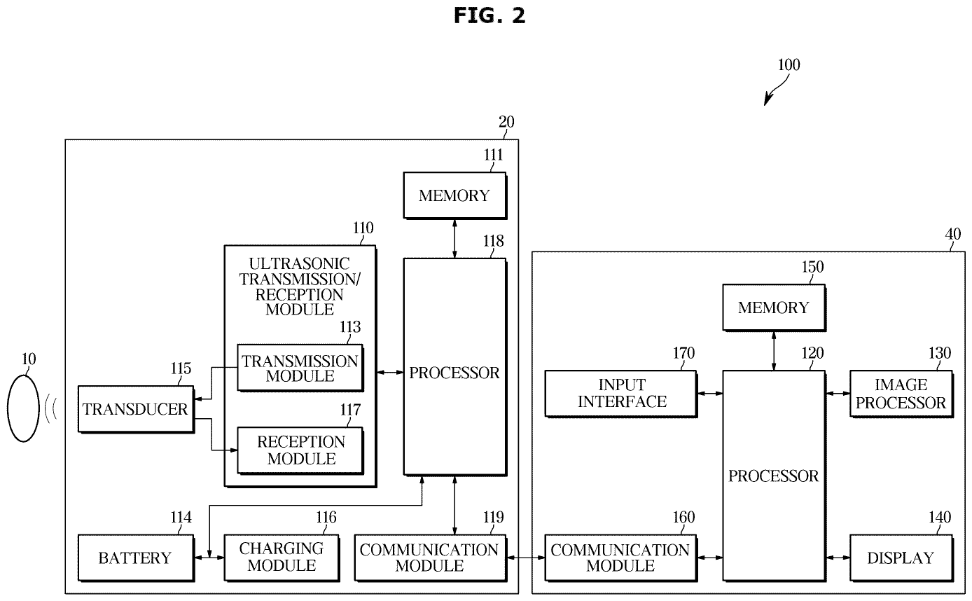

According to various embodiments, as illustrated in FIG. 1, the ultrasonic imaging apparatus 40 may include an ultrasonic transmission/reception module 110, and as illustrated in FIG. 2, the probe 20 may include the ultrasonic transmission/reception module 110. According to various embodiments, both the ultrasonic imaging apparatus 40 and the probe 20 may also include the ultrasonic transmission/reception module 110.

According to various embodiments, the probe 20 may further include an image processor 130, a display 140, and/or an input interface 170.

Accordingly, a description of the ultrasonic transmission/reception module 110, the image processor 130, the display 140, and/or the input interface 170 included in the ultrasonic imaging apparatus 40 may also be applied to the ultrasonic transmission/reception module 110, the image processor 130, the display 140, and/or the input interface 170 included in the probe 20.

FIG. 1 is a control block diagram of the ultrasonic imaging system 100 in a case in which the probe 20 is a wired probe or a hybrid probe.

The probe 20 may include a plurality of elements 115. The plurality of elements 115 may transmit an ultrasonic signal to an object 10 in response to a transmission signal applied from a transmission module 113. The plurality of elements 115 may form a reception signal by receiving the ultrasonic signal (echo signal) reflected from the object 10. The probe 20 may be implemented as an integrated type with the ultrasonic imaging apparatus 40, or may be implemented as a separate type connected to the ultrasonic imaging apparatus 40 by wire. The ultrasonic imaging apparatus 40 may be connected to the one or more probes 20 depending on the implementation type.

In the case in which the probe 20 is a wired probe or a hybrid probe, the probe 20 may include a cable and a connector connectable to a connector of the ultrasonic imaging apparatus 40.

The probe 20 according to an embodiment may be implemented as a two-dimensional probe. In a case in which the probe 20 is implemented as a two-dimensional probe, the plurality of elements 115 included in the probe 20 may be arranged in two dimensions to form a two-dimensional element array.

For example, the two-dimensional element array may have a form in which a plurality of sub-arrays including the plurality of elements 115 arranged in a first direction is arranged in a second direction different from the first direction.

Also, in the case in which the probe 20 according to an embodiment is implemented as a two-dimensional probe, the ultrasonic transmission/reception module 110 may include an analog beamformer and a digital beamformer. Alternatively, the two-dimensional probe may include one or both of the analog beamformer and the digital beamformer depending on the implementation type.

A processor 120 controls the transmission module 113 to form a transmission signal to be applied to each of the elements 115 in consideration of positions and focused points of the plurality of elements 115 included in the probe 20.

The processor 120 may control a reception module 117 to generate ultrasonic data by converting reception signals received from the probe 20 into analog to digital and summing up the digitally converted reception signals in consideration of the positions and focused points of the plurality of elements 115.

In the case in which the probe 20 is implemented as a two-dimensional probe, the processor 120 may calculate a time delay value for digital beamforming by each of the elements 115 for each of the plurality of elements 115 included in the two-dimensional element array. The processor 120 may also calculate a time delay value for analog beamforming for each of the elements 115 included in one of a plurality of the element arrays. The processor 120 may control the analog beamformer and the digital beamformer to form a transmission signal to be applied to each of the plurality of elements 115 depending on the time delay values for analog beamforming and the time delay values for digital beamforming. The processor 120 may also control the analog beamformer to sum up the signals received from the plurality of elements 115 by each sub-array depending on the time delay values for analog beamforming. The processor 120 may also control the ultrasonic transmission/reception module 110 to convert the summed signal by each sub-array into analog to digital. The processor 120 may also control the digital beamformer to generate ultrasonic data by summing up the digitally converted signals depending on the time delay values for digital beamforming.

The image processor 130 generates an ultrasonic image using the generated ultrasonic data.

The display 140 may display the generated ultrasonic image and a variety of information processed in the ultrasonic imaging apparatus 40 and/or the probe 20. The probe 20 and/or the ultrasonic imaging apparatus 40 may include the one or more displays 140 depending on the implementation type. The display 140 may also include a touch panel or a touch screen.

The display 140 may output a four-dimensional ultrasonic image in response to a control command from the processor 120. The four-dimensional ultrasonic image may refer to providing three-dimensional images in real time by adding the dimension of time. For example, the four-dimensional ultrasonic image may be an ultrasonic image including movement and heartbeat of a fetus or time-dependent movements of living tissue. The four-dimensional ultrasonic image may be implemented based on ultrasonic image data obtained in real time or ultrasonic image data previously stored in memory 150.

The processor 120 may control the overall operations of the ultrasonic imaging apparatus 40 and signal flows between internal components of the ultrasonic imaging apparatus 40. The processor 120 may perform or control various operations or functions of the ultrasonic imaging apparatus 40 by executing programs or instructions stored in the memory 150. The processor 120 may also control an operation of the ultrasonic imaging apparatus 40 by receiving a control signal from the input interface 170 or an external device.

The ultrasonic imaging apparatus 40 may include a communication module 160, and may be connected to an external device (e.g., the probe 20, a server, a medical device, a portable device (a smart phone, tablet PC, wearable device, etc.)) through the communication module 160.

The communication module 160 may include one or more components enabling communication with an external device and, may include, for example, at least one of a short-range communication module, a wired communication module, or a wireless communication module.

The communication module 160 may also receive a control signal and data from the external device and transmit the received control signal to the processor 120 so that the processor 120 may control the ultrasonic imaging apparatus 40 in response to the received control signal.

Alternatively, the processor 120 may transmit a control signal to the external device through the communication module 160, so that the external device may be controlled according to the control signal of the processor 120.

For example, the external device may process data in the external device according to the control signal of the processor 120 received through the communication module.

A program capable of controlling the ultrasonic imaging apparatus 40 may be installed in the external device, and the program may include instructions for performing part or all of the operations of the processor 120.

The program may be pre-loaded in the external device, or a user of the external device may download and install the program from a server providing an application. The server providing the application may include a storage medium in which the program is stored.

The memory 150 may store various data or programs for driving and controlling the ultrasonic imaging apparatus 40, inputted and outputted ultrasonic data, ultrasonic images, and the like.

The input interface 170 may receive user input for controlling the ultrasonic imaging apparatus 40. For example, the user input may include, but is not limited to, input of manipulating a button, a keypad, a mouse, a trackball, a jog switch, a knob, and the like, input of touching a touch pad or touch screen, voice input, motion input, biometric information input (e.g., iris recognition, fingerprint recognition, etc.), and the like.

FIG. 2 illustrates a control block diagram of the ultrasonic imaging system 100 in a case in which the probe 20 is a wireless probe or a hybrid probe.

According to various embodiments, the ultrasonic imaging apparatus 40 illustrated in FIG. 2 may be replaced with the ultrasonic imaging apparatus 40 described with reference to FIG. 1.

According to various embodiments, the probe 20 described with reference to FIG. 1 may be replaced with the probe 20 to be described with reference to FIG. 2.

The probe 20 may include the transmission module 113, a battery 114, the element 115, a charging module 116, the reception module 117, a processor 118, and a communication module 119. FIG. 2 illustrates that the probe 20 includes both the transmission module 113 and the reception module 115, but the probe 20 may include only part of configurations of the transmission module 113 and the reception module 117 depending on the implementation type, and the part of the configurations of the transmission module 113 and the reception module 117 may be included in the ultrasonic imaging apparatus 40. Additionally, the probe 20 may further include the image processor 130.

The transmission module 113 may include a transmission channel C1 provided to generate a transmission signal based on a synchronization signal. The transmission module 113 may include a plurality of the transmission channels C1. The synchronization signal, which is a signal having a pulse repetition frequency (PRF), may be referred to as a reference signal.

The reception module 117 may include a reception channel C2 provided to receive and process an echo signal received by the element 115. The reception module 117 may include a plurality of the reception channels C2.

The probe 20 may include the plurality of elements 115. The plurality of elements may transmit ultrasonic signals to the object 10 in response to transmission signals applied from the transmission module 113. The plurality of elements 115 may receive ultrasonic signals reflected from the object 10 to form reception signals.

The charging module 116 may charge the battery 114. The charging module 116 may receive electric power from the outside. The charging module 116 may receive electric power wirelessly. However, the charging module 116 is not limited thereto, and may also receive electric power by wire. The charging module 116 may transfer the received electric power to the battery 114.

The processor 118 controls the transmission module 113 to form a transmission signal to be applied to each of the plurality of elements 115 in consideration of the positions and focused points of the plurality of elements 115.

The processor 118 controls the reception module 117 to generate ultrasonic data by converting reception signals received from the elements 115 into analog to digital and summing up the digitally converted reception signals in consideration of the positions and focused points of the plurality of elements 115. Alternatively, in a case in which the probe 20 includes the image processor 130, the probe 20 may generate an ultrasonic image using the generated ultrasonic data.

In the case in which the probe 20 is implemented as a two-dimensional probe, the processor 118 may calculate the time delay value for digital beamforming by each sub-array for each of the plurality of elements 115 included in the two-dimensional element array. The processor 118 may also calculate the time delay value for analog beamforming for each of the elements 115 included in one of the plurality of element arrays. The processor 118 may control the analog beamformer and the digital beamformer to form a transmission signal to be applied to each of the plurality of elements 115 depending on the time delay values for analog beamforming and the time delay values for digital beamforming. The processor 118 may also control the analog beamformer to sum up the signals received from the plurality of elements 115 by each sub-array depending on the time delay values for analog beamforming. The processor 118 may also control the ultrasonic transmission/reception module 110 to convert the summed signal by each sub-array into analog to digital. The processor 118 may also control the digital beamformer to generate ultrasonic data by summing up the digitally converted signals depending on the time delay values for digital beamforming.

The processor 118 may control the overall operations of the probe 20 and signal flows between internal components of the probe 20. The processor 118 may perform or control various operations or functions of the probe 20 by executing programs or instructions stored in memory 111. The processor 118 may also control an operation of the probe 20 by receiving a control signal from the input interface 170 of the probe 20 or an external device (e.g., the ultrasonic imaging apparatus 40).

The communication module 119 may wirelessly transmit the generated ultrasonic data or ultrasonic images to the ultrasonic imaging apparatus 40 through a wireless network. The communication module 119 may also receive a control signal and data from the ultrasonic imaging apparatus 40.

The ultrasonic imaging apparatus 40 may receive the ultrasonic data or ultrasonic images from the probe 20.

In an embodiment, the case in which the probe 20 includes the image processor 130 capable of generating ultrasonic images using the ultrasonic data, the probe 20 may transmit the ultrasonic data and/or the ultrasonic images generated by the image processor 130 to the ultrasonic imaging apparatus 40.

In an embodiment, a case in which the probe 20 does not include the image processor 130 capable of generating ultrasonic images using the ultrasonic data, the probe 20 may transmit the ultrasonic data to the ultrasonic imaging apparatus 40. The ultrasonic data may include ultrasonic raw data, and the ultrasonic images may refer to ultrasonic image data.

The ultrasonic imaging apparatus 40 may include the processor 120, the image processor 130, the display 140, the memory 150, the communication module 160, and the input interface 170.

The image processor 130 generates ultrasonic images using the ultrasonic data received from the probe 20.

The display 140 may display the ultrasonic images received from the probe 20, ultrasonic images generated by processing the ultrasonic data received from the probe 20, and a variety of information processed in the ultrasonic imaging system 100. The ultrasonic imaging apparatus 40 may include the one or more displays 140 depending on the implementation type. The display 140 may also include a touch panel or a touch screen.

The processor 120 may control the overall operations of the ultrasonic imaging apparatus 40 and signal flows between the internal components of the ultrasonic imaging apparatus 40. The processor 120 may perform or control the various operations or functions of the ultrasonic imaging apparatus 40 by executing the programs or applications stored in the memory 150. The processor 120 may also control the operation of the ultrasonic imaging apparatus 40 by receiving a control signal from the input interface 170 or an external device.

The ultrasonic imaging apparatus 40 may include the communication module 160, and may be connected with an external device (e.g., the probe 20, a server, a medical device, a portable device (a smart phone, tablet PC, wearable device, etc.)) through the communication module 160.

The communication module 160 may include one or more components that enable communication with the external device, and may include, for example, at least one of a short-range communication module, a wired communication module, or a wireless communication module.

The communication module 160 of the ultrasonic imaging apparatus 40 and the communication module 119 of the probe 20 may communicate using a network or a short-range wireless communication method. For example, the communication module 160 of the ultrasonic imaging apparatus 40 and the communication module 119 of the probe 20 may communicate using any one of wireless LAN, Wi-Fi, Bluetooth, ZigBee, Wi-Fi Direct (WFD), Infrared Data Association (IrDA), Bluetooth Low Energy (BLE), Near Field Communication (NFC), Wireless Broadband Internet (WiBro), World Interoperability for Microwave Access (WiMAX), Shared Wireless Access Protocol (SWAP), Wireless Gigabit Alliance (WiGig), RF communication, and a wireless data communication method including 60 GHz millimeter wave (mm wave) short-range communication.

To this end, the communication module 160 of the ultrasonic imaging apparatus 40 and the communication module 119 of the probe 20 may include at least one of a wireless LAN communication module, a Wi-Fi communication module, a Bluetooth communication module, a ZigBee communication module, a Wi-Fi Direct (WFD) communication module, an Infrared Data Association (IrDA) communication module, a Bluetooth Low Energy (BLE) communication module, a Near Field Communication (NFC) module, a Wireless Broadband Internet (WiBro) communication module, a World Interoperability for Microwave Access (WiMAX) communication module, a Shared Wireless Access Protocol (SWAP) communication module, a Wireless Gigabit Alliance (WiGig) communication module, a RF communication module, or a 60 GHz millimeter wave (mm wave) short-range communication module.

In an embodiment, the probe 20 may transmit device information (e.g., ID information) of the probe 20 using a first communication method (e.g., BLE), may be wirelessly paired with the ultrasonic imaging apparatus 40, and may transmit ultrasonic data and/or ultrasonic images to the paired ultrasonic imaging apparatus 40.

The device information of the probe 20 may include a variety of information related to a serial number, model name, battery state of the probe 20, and the like.

The ultrasonic imaging apparatus 40 may receive the device information (e.g., ID information) of the probe 20 from the probe 20 using the first communication method (e.g., BLE), may be wirelessly paired with the probe 20, may transmit an activation signal to the paired probe 20, and may receive the ultrasonic data and/or ultrasonic images from the probe 20. In this case, the activation signal may include a signal for controlling the operation of the probe 20.

In an embodiment, the probe 20 may transmit the device information (e.g., ID information) of the probe 20 using the first communication method (e.g., BLE), may be wirelessly paired with the ultrasonic imaging apparatus 40, and may transmit the ultrasonic data and/or ultrasonic images to the ultrasonic imaging apparatus 40 paired by the first communication method using a second communication method (e.g., 60 GHz millimeter wave, Wi-Fi).

The ultrasonic imaging apparatus 40 may receive the device information (e.g., ID information) of the probe 20 from the probe 20 using the first communication method (e.g., BLE), may be wirelessly paired with the probe 20, may transmit the activation signal to the paired probe 20, and receive the ultrasonic data and/or ultrasonic images from the probe 20 using the second communication method (e.g., 60 GHz millimeter wave, Wi-Fi).

According to various embodiments, the first communication method used to pair the probe 20 and the ultrasonic imaging apparatus 40 with each other may have a frequency band lower than a frequency band of the second communication method used by the probe 20 to transmit the ultrasonic data and/or ultrasonic images to the ultrasonic imaging apparatus 40.

The display 140 of the ultrasonic imaging apparatus 40 may display UIs (user interfaces) indicating the device information of the probe 20. For example, the display 140 may display UIs, which indicate identification information of the wireless ultrasonic probe 20, a pairing method indicating a pairing method with the probe 20, a data communication state between the probe 20 and the ultrasonic imaging apparatus 40, a method of performing data communication with the ultrasonic imaging apparatus 40, and the battery state of the probe 20.

In a case in which the probe 20 includes the display 140, the display 140 of the probe 20 may display the UIs indicating the device information of the probe 20. For example, the display 140 may display the UIs, which indicate the identification information of the wireless ultrasonic probe 20, the pairing method indicating the pairing method with the probe 20, the data communication state between the probe 20 and the ultrasonic imaging apparatus 40, the method of performing the data communication with the ultrasonic imaging apparatus 40, and the battery state of the probe 20.

The communication module 160 may also receive a control signal and data from an external device and transmit the received control signal to the processor 120 so that the processor 120 may control the ultrasonic imaging apparatus 40 in response to the received control signal.

Alternatively, the processor 120 may transmit a control signal to an external device through the communication module 160, so that the external device may be controlled according to the control signal of the processor 120.

For example, the external device may process data in the external device according to the control signal of the processor 120 received through the communication module.

The program capable of controlling the ultrasonic imaging apparatus 40 may be installed in the external device, and the program may include instructions for performing part or all of the operations of the processor 120.

The program may be pre-loaded in the external device, or the user of the external device may download and install the program from the server providing the application. The server providing the application may include the storage medium in which the program is stored.

The memory 150 may store various data or programs for driving and controlling the ultrasonic imaging apparatus 40, inputted and outputted ultrasonic data, ultrasonic images, and the like.

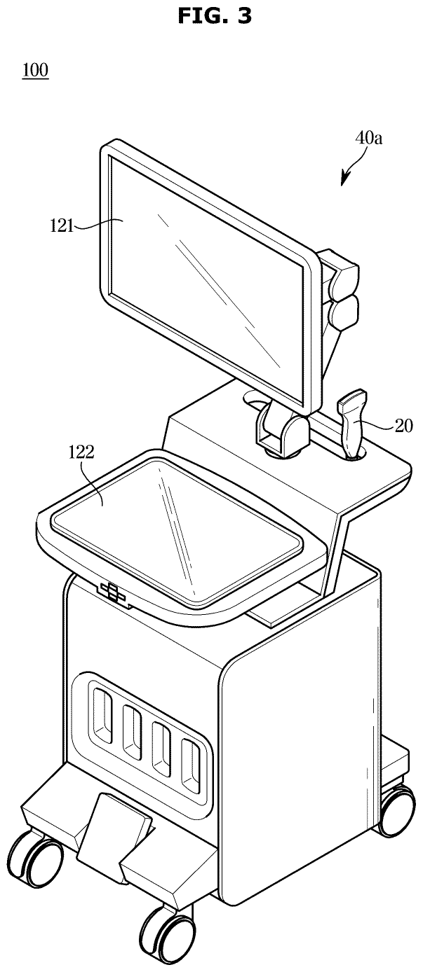

Examples of the ultrasonic imaging system 100 according to an embodiment of the disclosure will be described later with reference to FIGS. 3 to 6.

FIGS. 3 to 6 are views illustrating ultrasonic imaging apparatuses according to an embodiment.

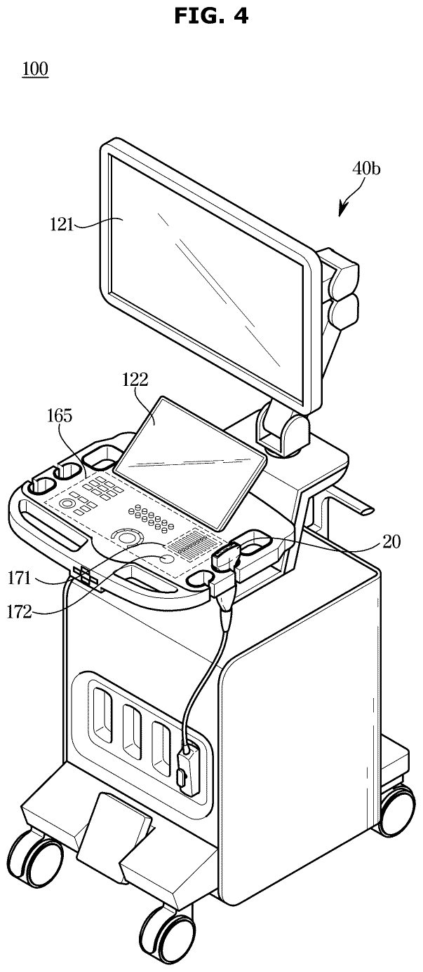

Referring to FIGS. 3 and 4, ultrasonic imaging apparatuses 40a and 40b may include a main display 121 (140) and a sub display 122 (140). At least one of the main display 121 or the sub display 122 may be implemented as a touch screen. At least one of the main display 121 or the sub display 122 may display ultrasonic images or a variety of information processed in the ultrasonic imaging apparatuses 40a and 40b. Also, at least one of the main display 121 or the sub display 122 may be implemented as a touch screen, and may receive input of data for controlling the ultrasonic imaging apparatuses 40a and 40b from a user by providing GUIs. For example, the main display 121 may display ultrasonic images, and the sub display 122 may display a control panel (e.g., a control panel 165 in FIG. 4) for controlling the display of the ultrasonic images in the form of GUIs. The sub display 122 may receive input of data for controlling the display of images through the control panel displayed in the form of GUIs. For example, a time gain compensation (TGC) button, a Freeze button, a trackball, a jog switch, a knob, and the like may be provided as GUIs on the sub display 122.

The ultrasonic imaging apparatuses 40a and 40b may control the display of ultrasonic images displayed on the main display 121 using the inputted control data. The ultrasonic imaging apparatuses 40a and 40b may be connected to the probe 20 by wire or wirelessly to transmit and receive ultrasonic signals to and from the object 10.

Referring to FIG. 4, the ultrasonic imaging apparatus 40b may further include a control panel 165 in addition to the main display 121 and the sub display 122. The control panel 165 may include a button, a trackball, a jog switch, a knob, and the like, and may receive input of data for controlling the ultrasonic imaging apparatus 40b from the user. For example, the control panel 165 may include a TGC button 171, a Freeze button 172, and the like. The TGC button 171 is a button for setting a TGC value by each of depths of ultrasonic images. The ultrasonic imaging apparatus 40b may keep a state in which a frame image at that point in time is displayed when the Freeze button 172 input is detected while scanning an ultrasonic image.

The button, trackball, jog switch, knob, and the like included in the control panel 165 may be provided as GUIs on the main display 121 or the sub display 122. The ultrasonic imaging apparatuses 40a and 40b may be connected to the probe 20 to transmit and receive ultrasonic signals to and from the object 10.

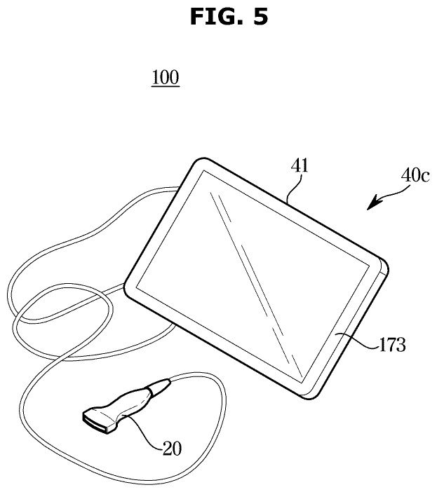

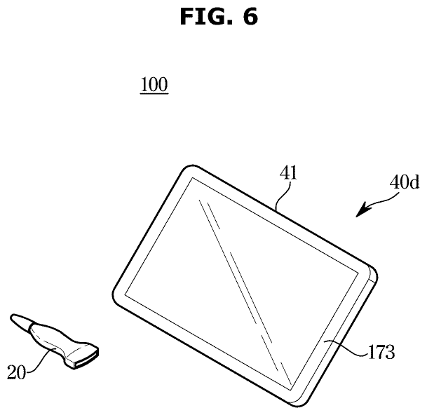

Referring to FIGS. 5 and 6, an ultrasonic imaging apparatus 40c may be implemented in a portable type. The portable ultrasonic imaging apparatus 40c may include, for example, a smart phone, a laptop computer, a PDA, a tablet PC, and the like which includes a probe and an application, but is not limited thereto.

The ultrasonic imaging apparatus 40c may include a main body 41. Referring to FIG. 5, the probe 20 may be connected to one side of the main body 41 by wire. To this end, the main body 41 may include a connection terminal to and from which a cable connected to the probe 20 may be attached and detached, and the probe 20 may include a connection terminal to and from which a cable connected to the main body 41 may be attached and detached.

Referring to FIG. 6, the probe 20 may be wirelessly connected to an ultrasonic imaging apparatus 40d. The main body 41 may include an input/output interface (e.g., a touch screen) 173 (140 and 170). Ultrasonic images, a variety of information processed in the ultrasonic imaging apparatus, GUIs, and the like may be displayed on the input/output interface 173.

Also, an ultrasonic image may be displayed on the input/output interface 173. The ultrasonic imaging apparatus 40c may correct the ultrasonic image displayed on the input/output interface 173 using AI. The ultrasonic imaging apparatus 40c may provide an alarm notifying information about a lesion in the ultrasonic image displayed on the input/output interface 173 through various audiovisual tools such as graphics, sounds, and vibrations by using the AI.

The ultrasonic imaging apparatus 40c may output a control panel displayed in the form of a GUI through the input/output interface 173.

The ultrasonic imaging apparatus 40d and the probe 20 may establish communication or be paired using a short-range wireless communication. For example, the ultrasonic imaging apparatus 40d and the probe 20 may perform communication using Bluetooth, BLE, Wi-Fi, or Wi-Fi Direct.

The ultrasonic imaging apparatuses 40c and 40d may execute a program or application related to the probe 20 to control the probe 20 and output information related to the probe 20. The ultrasonic imaging apparatuses 40c and 40d may perform operations related to the probe 20 while communicating with a predetermined server. The probe 20 may be registered with the ultrasonic imaging apparatuses 40c and 40d or may be registered with the predetermined server. The ultrasonic imaging apparatuses 40c and 40d may communicate with the registered probe 20 and perform the operations related to the probe 20.

The ultrasonic imaging apparatuses 40c and 40d may also include various types of input/output interfaces such as speakers, LEDs, and vibration devices. For example, the ultrasonic imaging apparatuses 40c and 40d may output a variety of information in the form of graphics, sound, or vibration through the input/output interfaces. The ultrasonic imaging apparatuses 40c and 40d may also output various notifications or data through the input/output interfaces.

According to an embodiment of the disclosure, the ultrasonic imaging apparatus 40a, 40b, 40c, or 40d may process an ultrasonic image or obtain additional information from the ultrasonic image using an artificial intelligence (AI) model. According to an embodiment of the disclosure, the ultrasonic imaging apparatus 40a, 40b, 40c, or 40d may, using the AI model, generate an ultrasonic image, or perform processing such as correction, image quality improvement, encoding, or decoding on an ultrasonic image. According to an embodiment of the disclosure, the ultrasonic imaging apparatus 40a, 40b, 40c, or 40d may also, using the AI model, perform processing of reference line definition, anatomical information obtainment, lesion information obtainment, surface extraction, boundary definition, length measurement, area measurement, volume measurement, or annotation creation, from an ultrasonic image.

The AI model may be provided on the ultrasonic imaging apparatus 40a, 40b, 40c, or 40d, or may be provided on the server.

The AI model may be implemented using various artificial neural network models or deep neural network models. In addition, the AI model may be learned and created using various machine learning algorithms or deep learning algorithms. The AI model may be implemented using, for example, a model such as a convolutional neural network (CNN), a recurrent neural network (RNN), a generative adversarial network (GAN), or a long short-term memory (LSTM).

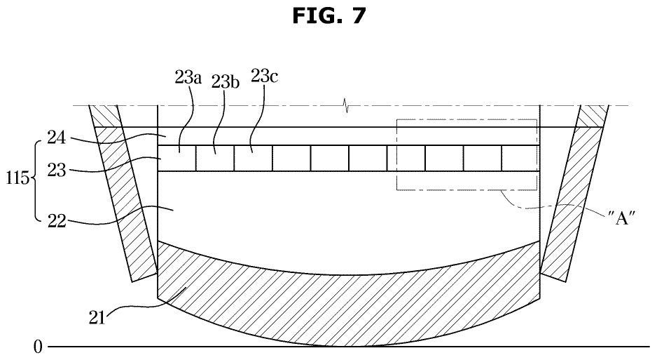

FIG. 7 is a view illustrating a portion of the probe 20 according to an embodiment.

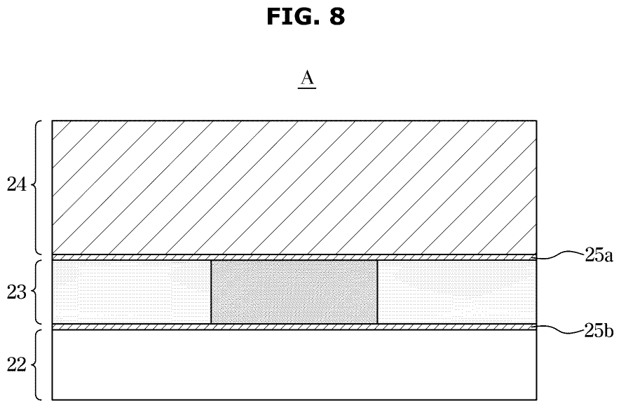

FIG. 8 is an enlarged view of a region A of the probe 20 illustrated in FIG. 7 according to an embodiment.

The probe 20 may irradiate ultrasonic waves to an object and receive echo ultrasonic waves reflected from the object.

According to one embodiment, the probe 20 includes a case configured to accommodate the plurality of elements 115. An examiner may perform an examination by holding the case and coming one surface of the probe 20 into contact with the object.

The probe 20 may include the plurality of elements 115 and an acoustic lens 21. The plurality of elements 115 may be implemented in the form of an array arranged side by side. The element 115 may include a matching layer 22, a piezoelectric element 23 and/or a sound-absorbing layer 24. The acoustic lens 21, the matching layer 22, the piezoelectric element 23, and the sound-absorbing layer 24 may be arranged sequentially from the front surface coming into contact with the object.

The acoustic lens 21 may be provided in front of the matching layer 22. The acoustic lens 21 concentrates ultrasonic signals traveling forward at a specific point (i.e., focus). As illustrated in FIG. 7, the acoustic lens 21 is provided in a convex shape, but is not limited thereto and may also be provided in a concave shape.

The matching layer 22 may be provided in front of the piezoelectric element 23. The matching layer 22 serves to match an acoustic impedance of the piezoelectric element 23 with an acoustic impedance of the object so that an ultrasonic signal generated from the piezoelectric element 23 is efficiently transmitted to the object. To this end, the matching layer 22 may be provided to have an intermediate value between the acoustic impedance of the piezoelectric element 23 and the acoustic impedance of the object.

The matching layer 22 may be made of glass or resin material. According to various embodiments, a plurality of the acoustic matching layers made of different materials may be provided such that the acoustic impedance may be changed stepwise from the piezoelectric element 23 toward the object.

The piezoelectric element 23 may be bonded to a front surface of the sound-absorbing layer 24. The piezoelectric element 23 may convert an electrical signal into an ultrasonic wave, which is an acoustic signal, emit the ultrasonic wave into the air, convert an echo signal reflected in the air back into an electrical signal, and transmit the converted electrical signal to the main body 40. The piezoelectric element 23 may be implemented in the form of an array in which a plurality of piezoelectric elements 23a, 23b, and 23c is arranged.

As illustrated in FIG. 8, a plurality of electrodes 25a and 25b included in an electrode unit 25 may be provided on both side surfaces of the piezoelectric element 23. The electrode unit (not shown) formed on the side surfaces of the piezoelectric element 23 may be formed of a highly conductive metal such as gold, silver, or copper, or graphite.

The piezoelectric elements 23a, 23b, and 23c may be implemented as piezoelectric materials generating ultrasonic waves using a resonance phenomenon. The piezoelectric material may be formed of a ceramic of lead zirconate titanate (PZT), a PNZT single crystal made from a solid solution of zinc lead niobate and lead titanate, and a PZMT single crystal made from a solid solution of magnesium lead niobate and lead titanate.

The sound-absorbing layer 24 may be provided at the rear of the piezoelectric element 23. The sound-absorbing layer 24 may reduce a pulse width of ultrasonic waves by suppressing free vibration of the piezoelectric element 23, and may block ultrasonic waves from propagating unnecessarily to the rear of the piezoelectric element 23. Accordingly, the sound-absorbing layer 24 may prevent distortion of ultrasonic images.

A printed circuit board (PCB) (not shown) may be positioned between the sound-absorbing layer 24 and the piezoelectric element 23. The printed circuit board (not shown) may mutually convert electrical signals and ultrasonic signals generated from the electrode unit 25. The printed circuit board (not shown) may be formed in a direction perpendicular to a lamination direction of the sound-absorbing layer 24 and the piezoelectric element 23. The printed circuit board (not shown) may include a component through which signals may supply electricity, such as a flexible printed circuit (FPCB).

In a manufacturing process of the probe 20, respective components such as the acoustic lens 21, the matching layer 22, the piezoelectric element 23, and the sound-absorbing layer 24 may have unique acoustic characteristics deviations due to variations in manufacturing processes and material properties of the respective components. These deviations may affect quality of an ultrasonic signal and accuracy of an ultrasonic image, and therefore correction is required.

According to an embodiment, a correction parameter (hereinafter referred to as a pre-correction parameter) for correcting a deviation in unique acoustic characteristics of the probe 20 may be obtained before the probe 20 is produced and connected to the main body 40, and then used.

The pre-correction parameter may include a first pre-correction sub-parameter for correcting deviations due to the unique characteristics of at least one of the acoustic lens 21, the matching layer 22, each of the plurality of piezoelectric elements 23a, 23b, 23c, etc., or the sound-absorbing layer 24, which constitute the probe 20.

The pre-correction parameter may include a second pre-correction sub-parameter for correcting an acoustic characteristics deviation due to the unique characteristics of the electrode unit 25 that transmits a transmission signal to the piezoelectric element 23.

According to an embodiment, the pre-correction parameter may be obtained before the probe 20 is produced and connected to the main body 40, and then used.

Specifically, data for obtaining the pre-correction parameter may be collected. In this case, the collected data may correspond to data reflecting the acoustic characteristics deviation due to the unique characteristics of the acoustic lens 21, matching layer 22, piezoelectric element 23, sound-absorbing layer 24, or electrode unit 25. For example, data may be collected by connecting the probe 20 to a jig, adjusting transmission parameters (e.g., frequency, waveform, depth, etc.) to transmit ultrasonic waves, and collecting reflected echo signals.

When data for obtaining the pre-correction parameter about the specific probe 20 is collected, the transmission parameter need to be adjusted for each of the plurality of elements 115 included in the specific probe 20. Therefore, when a plurality of the transmission parameters is adjusted under various conditions, more calculations and time may be required. For example, when, among the transmission parameters, the depth is to be adjusted to three conditions (1 cm, 5 cm, and 10 cm), the frequency to be adjusted to three conditions (1 MHz, 5 MHz, and 10 MHz), and the waveform to be adjusted to three conditions, the transmission parameters need to be adjusted to 3×3×3 conditions, that is, a total of 27 conditions. In this case, when the probe 20 includes the 192 elements 115, a massive calculation of as much as 27×192 is required.

According to an embodiment, before the probe 20 is produced and connected to the main body 40, and then used, the pre-correction parameter may be obtained by inputting data collected under various transmission conditions of various transmission parameters into an artificial intelligence model.

According to an embodiment, after data is collected, an artificial intelligence learning model may be established. Specifically, collected data (i.e., raw data) may be inputted to the artificial intelligence model, a phase deviation may be calculated by comparing a reference signal and the collected data, and a parameter for correcting the calculated phase deviation may be obtained. Thereafter, a magnitude deviation may be calculated by comparing the reference signal and the collected data, and a parameter for correcting the calculated magnitude deviation may be obtained.

Thereafter, the pre-correction parameter may be calculated using the parameter for correcting the obtained phase deviation and the parameter for correcting the magnitude deviation. Accordingly, rapid correction may be performed without large-scale calculations. The calculated pre-correction parameter may be stored in the memory of the probe 20.

The artificial intelligence model for obtaining the pre-correction parameter may utilize optimization algorithms such as Stochastic Gradient Descent (SGD), Adam, RMSProp, etc. Also, the artificial intelligence model may obtain the pre-correction parameter through a method of minimizing an error between a predicted value of the model and an actual measured value through a loss function.

After the probe 20 is mounted on the main body 40, a transmission delay time or a transmission intensity may be corrected in order to correct the phase deviation and/or the magnitude deviation using the pre-correction parameter stored in the probe 20. Specifically, the artificial intelligence model may be used to correct the transmission delay time or the transmission intensity. For example, when the first pre-correction sub-parameter and one of the 27×192 transmission parameters are inputted to the artificial intelligence model, a transmission signal with the correspondingly corrected transmission delay time or transmission intensity may be outputted. That is, when conditions of 1 cm depth, 5 MHz frequency, and second waveform are inputted into the artificial intelligence model along with the first pre-correction sub-parameter, a transmission signal with the correspondingly corrected transmission delay time or transmission intensity may be outputted.

In this case, an artificial intelligence model for correcting the transmission delay time or transmission intensity may be implemented as the same model as the artificial intelligence model for obtaining the pre-correction parameter, or may be implemented as a different model.

That is, by inputting a specific transmission parameter and the pre-correction parameter into the artificial intelligence model, ultrasonic waves may be generated in which deviations in acoustic characteristics due to the unique characteristics of the acoustic lens 21 of the probe 20, the matching layer 22, each of the plurality of piezoelectric elements 23a, 23b, 23c, etc., the sound-absorbing layer 24, or the electrode unit 25 are corrected.

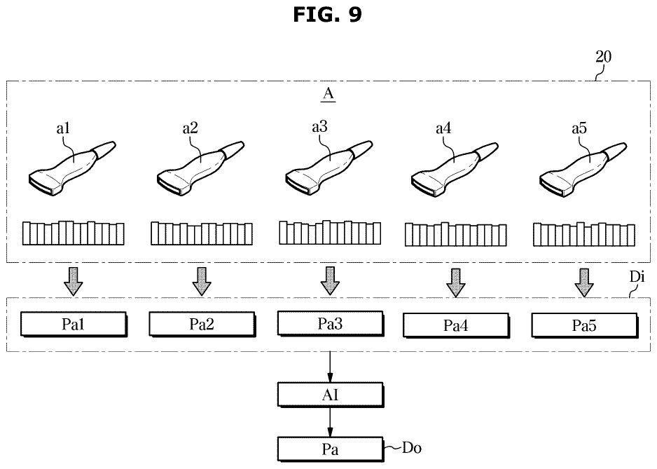

FIG. 9 is a diagram for explaining a method of obtaining a third pre-correction sub-parameter for correcting a deviation in acoustic characteristics.

According to an embodiment, the pre-correction parameter may include the third pre-correction sub-parameter for correcting an acoustic characteristics deviation due to unique characteristics of a type of the probe 20.

The third pre-correction sub-parameter may be a sub-parameter obtained as input data a plurality of acoustic characteristic pre-correction parameters corresponding to the respective probes 20 of the same type as the probes 20 using the artificial intelligence model.

Specifically, referring to FIG. 9, unique data for each of a plurality of the probes 20 belonging to the same type may be collected in order to obtain the third pre-correction sub-parameter using the artificial intelligence model.

For example, a first probe a1, a second probe a2, a third probe a3, a fourth probe a4, and a fifth probe a5 may correspond to the same type of the probe 20.

The probe a1, the second probe a2, the third probe a3, the fourth probe a4, and the fifth probe a5 may have different acoustic characteristics deviations. Accordingly, the first probe a1, the second probe a2, the third probe a3, the fourth probe a4, and the fifth probe a5 may have individual pre-correction parameters to correct the acoustic characteristics deviations.

The collecting of the unique data for each of the plurality of probes 20 may include collecting data about pre-correction parameters of the individual probes a1, a2, a3, a4, and a5 included in the type of the probe 20. In this case, the collected pre-correction parameter may include the above-described first pre-correction sub-parameter and/or second pre-correction sub-parameter.

For example, data Pa1 about the pre-correction parameter of the first probe a1, data Pa2 about the pre-correction parameter of the second probe a2, data Pa3 about the pre-correction parameter of the third probe a3, data Pa4 about the pre-correction parameter of the fourth probe a4, and data Pa5 about the pre-correction parameter of the fifth probe a5 may be collected. These data reflect the unique characteristics of the individual probes a1, a2, a3, a4, and a5, and based on this, the third pre-correction sub-parameter for correcting an acoustic deviation common to the types of the probe 20 may be obtained.

In this case, the data Pa1, Pa2, Pa3, Pa4, and Pa5 about the pre-correction parameters of the collected individual probes a1, a2, a3, a4, and a5 may be used as input data Di of the artificial intelligence model. For example, an artificial intelligence model AI may analyze correlation between the data Pa1, Pa2, Pa3, Pa4, and Pa5 about the pre-correction parameters of the individual probes a1, a2, a3, a4, and a5. Accordingly, the third pre-correction sub-parameter Pa, which is commonly applicable to the types of the probe 20 including the individual probes a1, a2, a3, a4, and a5, may be derived. That is, the third pre-correction sub-parameter Pa, which is commonly applicable to the types of the probe 20, may be obtained as output data Do by utilizing the artificial intelligence model.

According to an embodiment, as the main body 40 and the probe 20 are connected, the pre-correction parameter obtained by the method described above may be transferred from the probe 20 to the main body 40. In this case, the pre-correction parameter may include the first pre-correction sub-parameter, the second pre-correction sub-parameter and/or the third pre-correction sub-parameter.

Specifically, the processor 120 may control the main body communication module 160 to receive the pre-correction parameter from the probe 20. As the main body 40 and the probe 20 are connected, the main body 40 may identify the probe 20. The main body 40 may obtain information about a unique identifier (e.g., a unique ID or serial number of the probe 20) through communication with the probe 20 connected to the main body 40, and identify the connected probe 20. Accordingly, the main body 40 may obtain the pre-correction parameter from the identified probe 20.

The main body 40 may obtain the pre-correction parameter about the identified probe 20 from an external device excluding the probe 20. In this case, the external device may include a server device, or a main body different from the main body 40 to which the probe 20 is connected. Specifically, the processor 120 may control the main body communication module 160 to receive the pre-correction parameter from an external device.

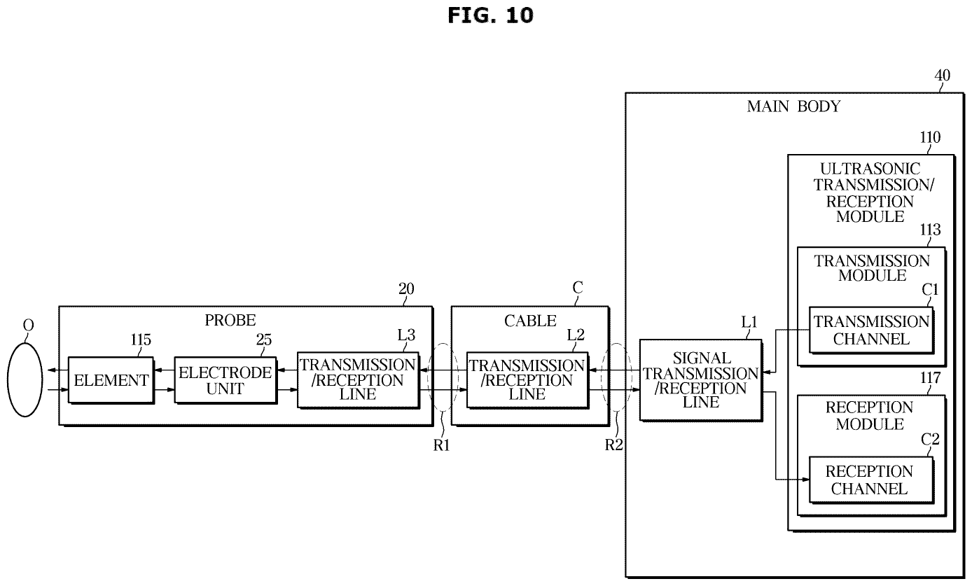

FIG. 10 is a diagram for explaining a post-correction parameter according to an embodiment.

According to an embodiment, the probe 20, a cable C, and the main body 40 may each include a signal transmission/reception line.

The main body 40 may include a signal transmission/reception line L1 configured to transmit a transmission signal generated by the transmission channel C1 included in the transmission module 113 to the element 115 of the probe 20 via the cable C and to transmit an echo signal received by the element 115 to the reception channel C2 included in the reception module 117 via the cable C. In this case, the transmitting of the transmission signal generated by the transmission channel C1 to the element 115 may include transmitting the transmission signal to the piezoelectric element 23 included in each of the plurality of elements 115. That is, the signal transmission/reception line L1 within the main body 40 may correspond to a path for the transmission signal and the echo signal. One end of the signal transmission/reception line L1 may be connected to the transmission channel C1 and the reception channel C2, and the other end may be connected to a transmission/reception line L2 of the cable C.

The cable C may include the signal transmission/reception line L2 configured to transmit a signal between the main body 40 and the probe 20. That is, one end of the signal transmission/reception line L2 within the cable C may be connected to the signal transmission/reception line L1 within the main body 40, and the other end may be connected to a signal transmission/reception line L3 within the probe 20.

The probe 20 may include the signal transmission/reception line L3 configured to transmit a transmission signal received from the signal transmission/reception line L2 within the cable C to the element 115 and to transmit an echo signal received by the element 115 to the signal transmission/reception line L2 within the cable C in order to transmit the echo signal to the reception channel C2. One end of the signal transmission/reception line L3 within the probe 20 may be connected to the signal transmission/reception line L2 within the cable C, and the other end may be connected to the electrode unit 25.

Acoustic characteristics deviations may occur when signals are transmitted through the signal transmission/reception line L1 within the main body 40, the signal transmission/reception line L2 within the cable C, and the signal transmission/reception line L3 within the probe 20. Specifically, acoustic characteristics deviations may occur due to factors such as signal attenuation caused by differences in a length and thickness of signal transmission/reception lines when a signal is transmitted, distortion caused by trace interference due to proximity of transmission and reception signals, or signal leakage (coupling). That is, acoustic characteristics deviations may occur in a process of transmitting signals after the probe 20 is connected to the main body 40. These deviations may affect the quality of ultrasonic signals and the accuracy of ultrasonic images, and therefore corrections are required.

According to an embodiment, correction parameters (hereinafter referred to as post-correction parameters) for correcting the acoustic characteristics deviations occurred in the process of transmitting and receiving signals after the probe 20 is connected to the main body 40 may be obtained.

According to an embodiment, in order to obtain the post-correction parameter, the probe 20 may receive an echo signal in which an ultrasonic wave with the acoustic characteristics deviation corrected by the pre-correction parameter is reflected from an object. The echo signal may be transmitted to the main body 40 through the signal transmission/reception lines L1, L2, and L3.

The processor 120 may control the reception channel C2 to obtain processed data by processing the echo signal.

The processor 120 may obtain the post-correction parameter for correcting the acoustic characteristics deviation occurred in the process of transmitting and receiving a signal between the probe 20 and the main body 40 by using the processed data as input data using the artificial intelligence model. For example, a model may be learned to minimize deviations by learning ideal data with the post-correction parameters reflected, i.e., target values, and then generate correction parameters. To this end, mapping relationships between the acoustic characteristics deviations occurred in the process of transmitting and receiving signals and the post-correction parameters may be learned using a supervised learning method.

The processor 120 may control the transmission channel C1 to correct the generated transmission signals based on the post-correction parameters.

Thereafter, the probe 20 may receive the corrected transmission signal, and generate and output an ultrasonic signal corrected in the element 115 based on the corrected transmission signal.

The post-correction parameter may include a first post-correction sub-parameter for correcting acoustic characteristics deviations due to unique characteristics of at least one of the signal transmission/reception line L1 within the main body 40, the signal transmission/reception line L2 within the cable C, or the signal transmission/reception line L3 within the probe 20.

The post-correction parameter may include a second post-correction sub-parameter for correcting an acoustic characteristics deviation due to a connection relationship between the signal transmission/reception line L1 within the main body 40 and the signal transmission/reception line L2 within the cable C or a connection relationship between the signal transmission/reception line L3 within the probe 20 and the signal transmission/reception line L2 within the cable C.

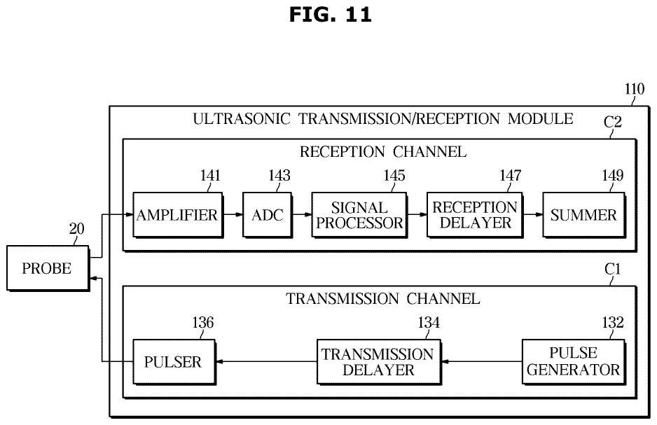

FIG. 11 is a diagram for explaining generating and/or correcting a transmission signal according to an embodiment.

The ultrasonic transmission/reception module 110 may include the transmission channel C1 and/or the plurality of reception channels C2. The probe 20 may output an ultrasonic signal to an object in response to a transmission signal applied from the ultrasonic transmission/reception module 110 and receive an echo signal reflected from the object.

The transmission channel C1 may include a pulse generator 132, a transmission delayer 134, and a pulser 136. The pulse generator 132 may generate pulses to form a transmission ultrasonic wave according to a predetermined pulse repetition frequency (PRF), and the transmission delayer 134 may apply a delay time for determining transmission directionality to the pulses. Each pulse to which the delay time is applied may be transmitted to each of the plurality of piezoelectric elements 23a, 23b, 23c, etc., included in the probe 20. The pulser 136 may apply a transmission signal to the probe 20 at timing corresponding to each pulse to which the delay time is applied. The pulser 136 my adjust an intensity (or amplitude) of the transmission signal.

According to various embodiments, the transmission channel C1 of the ultrasonic transmission/reception module 110 may be configured as a plurality of the transmission channels C1 connected to the plurality of piezoelectric elements 23a, 23b, 23c, etc., included in the element 115. Accordingly, the transmission signal may be transmitted to the piezoelectric materials through each of the transmission channels C1. According to various embodiments, any one of the plurality of transmission channels C1 may transmit the transmission signal to one or more of the piezoelectric elements 23a, 23b, 23c, etc.

The reception channel C2 may include an amplifier 141, an analog digital converter (ADC) 143, a signal processor 145, a reception delayer 147, and/or a summer 149. The amplifier 141 may amplify an echo signal for each of the reception channels, and the ADC 143 may convert the amplified echo signal into analog-digital. The signal processor 145 may remove noise, leaving only an effective frequency band, by utilizing a low pass filter, a high pass filter, etc. The reception delayer 147 may apply a delay time for determining reception directionality to the digitally converted echo signals, and the summer 149 may generate ultrasonic data by summing the echo signals processed by the reception delayer 147. The reception channel C2 may be implemented in a form in which at least one part of the above-described configuration is omitted. For example, when sensitivity of the probe 20 is improved or the number of processing bits of the ADC 143 is improved, the amplifier 141 may be omitted.

According to an embodiment, the processor 120 may determine a transmission parameter based on at least one of an object or a diagnosis subject. The object or diagnostic subject may be determined depending on user input. In this case, the transmission parameter may include at least one of a depth, frequency, or waveform.

When the transmission parameter is determined, the processor 120 may correct a preset transmission delay time or a preset transmission intensity corresponding to the transmission parameter based on the pre-correction parameter. In this case, the preset transmission delay time or the preset transmission intensity corresponding to the transmission parameter may be stored in the memory 150. The correcting of the transmission delay time based on the pre-correction parameter may include controlling the transmission delayer 134 based on the pre-correction parameter by the processor 120. Also, the correcting of the transmission intensity based on the pre-correction parameter may include controlling the pulser 136 based on the pre-correction parameter by the processor 120. Accordingly, the corrected transmission signal may be transmitted to the probe 20. Accordingly, the probe 20 may generate an ultrasonic signal based on the corrected transmission signal.

Additionally, when the post-correction parameter is obtained, the processor 120 may correct the transmission signal generated based on the post-correction parameter. In this case, the correcting of the generated transmission signal based on the post-correction parameter may include controlling the transmission delayer 134 and/or the pulser 136 based on the post-correction parameter by the processor 120.

Although the disclosure has described a case in which the ultrasonic transmission/reception module 110 is included in the main body 40 as an example, the ultrasonic transmission/reception module 110 may be provided in the probe 20 according to various embodiments.

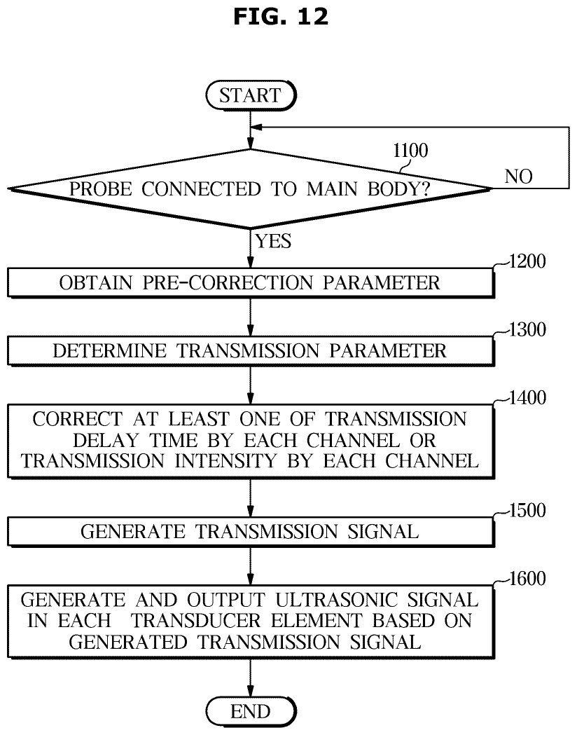

FIG. 12 is a flowchart of a method of correcting an acoustic characteristics deviation using a pre-correction parameter according to an embodiment.

An acoustic characteristics correction method of the ultrasonic imaging apparatus 100 according to an embodiment may include determining whether the main body 40 and the probe 20 are connected (1100).

The acoustic characteristics correction method of the ultrasonic imaging apparatus 100 may include obtaining a pre-correction parameter about the probe 20 (1200) when the main body 40 and the probe 20 are connected (YES in 1100). The pre-correction parameters may be previously obtained using an artificial intelligence model and stored in the memory 111 of the probe 20. For example, the processor 120 may control the communication module 160 to receive the pre-correction parameters from the probe 20 connected to the main body 40. As another example, the processor 120 may control the communication module 160 to receive the pre-correction parameters from an external device. In this case, the external device may include a server device and/or a main body different from the main body 40 to which the probe 20 is connected.

In this case, the pre-correction parameter may include the first pre-correction sub-parameter for correcting deviations due to the unique characteristics of at least one of the acoustic lens 21, the matching layer 22, plurality of piezoelectric elements 23a, 23b, 23c, etc., or the sound-absorbing layer 24, which constitute the probe 20.

The pre-correction parameter may include the second pre-correction sub-parameter for correcting the acoustic characteristics deviation due to the unique characteristics of the electrode unit 25 that transmits the transmission signal to the piezoelectric element 23.

The pre-correction parameter may include the third pre-correction sub-parameter for correcting the acoustic characteristics deviation due to the unique characteristics of the type of the probe 20.

The acoustic characteristics correction method of the ultrasonic imaging apparatus 100 may include determining a transmission parameter based on at least one of an object or a diagnostic subject (1300). In this case, the transmission parameter may include at least one of the depth, frequency, or waveform. The object or the diagnostic subject may be determined depending on the user input.

The acoustic characteristics correction method of the ultrasonic imaging apparatus 100 may include correcting at least one of the preset transmission delay time or the preset transmission intensity corresponding to the transmission parameter based on the pre-correction parameter (1400). The processor 120 may correct the transmission delay time by controlling the transmission delayer 134 based on the pre-correction parameter. Also, the processor 120 may correct the transmission intensity by controlling the pulser 136 based on the pre-correction parameter.

The acoustic characteristics correction method of the ultrasonic imaging apparatus 100 may include controlling the transmission channel C1 to generate a transmission signal based on at least one of the corrected transmission delay time or the corrected transmission intensity (1500).

The acoustic characteristics correction method of the ultrasonic imaging apparatus 100 may include generating and outputting an ultrasonic signal corrected in the element 115 connected to the transmission channel C1 based on the generated transmission signal (1600).

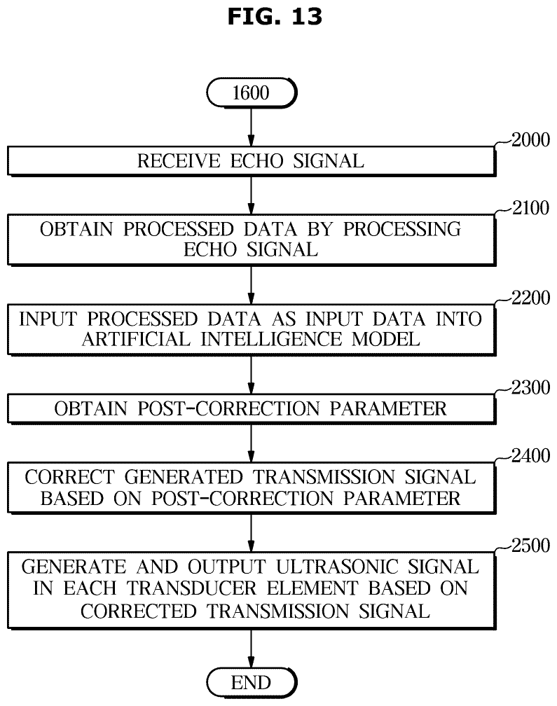

FIG. 13 is a flowchart of a method of correcting an acoustic characteristics deviation using a post-correction parameter according to an embodiment.

An acoustic characteristics correction method of the ultrasonic imaging apparatus 100 according to an embodiment may include receiving an echo signal in which the ultrasonic signal generated by the element 115 connected to the transmission channel C1 is reflected from an object based on the generated transmission signal (2000).

The acoustic characteristics correction method of the ultrasonic imaging apparatus 100 may include obtaining processed data by processing the echo signal (2100). The processor 120 may control the reception channel C2 to obtain the processed data by processing the echo signal.

The acoustic characteristics correction method of the ultrasonic imaging apparatus 100 may include inputting the processed data as input data into the artificial intelligence model (2200).

Thereafter, the acoustic characteristics correction method of the ultrasonic imaging apparatus 100 may include obtaining a post-correction parameter from the artificial intelligence model (2300). The post-correction parameter may correspond to a correction parameter for correcting an acoustic characteristics deviation occurred in the process of transmitting and receiving a signal between the probe 20 and the main body 40.

The post-correction parameter may include the first post-correction sub-parameter for correcting the acoustic characteristics deviations due to unique characteristics of at least one of the signal transmission/reception line L1 within the main body 40, the signal transmission/reception line L2 within the cable C, or the signal transmission/reception line L3 within the probe 20.

The post-correction parameter may include the second post-correction sub-parameter for correcting an acoustic characteristics deviation due to the connection relationship between the signal transmission/reception line L1 within the main body 40 and the signal transmission/reception line L2 within the cable C or the connection relationship between the signal transmission/reception line L3 within the probe 20 and the signal transmission/reception line L2 within the cable C.

The acoustic characteristics correction method of the ultrasonic imaging apparatus 100 may include correcting the generated transmission signal based on the post-correction parameter (2400). The processor 120 may control the transmission delayer 134 and/or the pulser 136 to correct the transmission signal based on the post-correction parameter.

The acoustic characteristics correction method of the ultrasonic imaging apparatus 100 may include generating and outputting an ultrasonic signal corrected in the element 115 connected to the transmission channel C1 based on the corrected transmission signal (2500).

An acoustic characteristics correction method of an ultrasonic imaging apparatus according to an embodiment, which includes a main body including a transmission channel configured to generate a transmission signal based on a synchronization signal, and a probe including a plurality of elements configured to generate an ultrasonic signal based on the transmission signal, may include obtaining a pre-correction parameter about the probe based on connection of the main body and the probe, determining a transmission parameter based on at least one of an object or a diagnostic subject, correcting at least one of a preset transmission delay time or a preset transmission intensity corresponding to the transmission parameter based on the pre-correction parameter, controlling the transmission channel to generate a transmission signal based on at least one of the corrected transmission delay time or the corrected transmission intensity, and generating and outputting an ultrasonic signal in the element connected to the transmission channel based on the generated transmission signal.

The obtaining of the pre-correction parameter may include obtaining a first pre-correction sub-parameter for correcting an acoustic characteristics deviation due to unique characteristics of at least one of a plurality of piezoelectric elements included in the element, an acoustic lens, a matching layer, or a sound-absorbing layer, which constitute the probe.

The obtaining of the pre-correction parameter may include obtaining a second pre-correction sub-parameter for correcting an acoustic characteristics deviation due to unique characteristics of an electrode unit configured to transmit the transmission signal to the plurality of piezoelectric elements.

The obtaining of the pre-correction parameter may include obtaining a third pre-correction sub-parameter for correcting an acoustic characteristics deviation due to unique characteristics of a type of the probe, and the third pre-correction sub-parameter may be a sub-parameter obtained as input data a plurality of acoustic characteristic pre-correction parameters corresponding to a plurality of respective probes of the same type as the probe using an artificial intelligence model.

The obtaining of the pre-correction parameter may include controlling a main body communication module and a probe communication module to receive the pre-correction parameter from the probe connected to the main body.

The ultrasonic imaging apparatus may further include a reception channel configured to receive and process echo signals received by the plurality of elements, and the acoustic characteristics correction method of the ultrasonic imaging apparatus may include receiving an echo signal reflected from the object, controlling the reception channel to obtain processed data by processing the echo signal, obtaining a post-correction parameter for correcting an acoustic characteristics deviation occurred in a process of transmitting and receiving a signal between the probe and the main body by using the processed data as input data using the artificial intelligence model, correcting the generated transmission signal based on the post-correction parameter, and generating and outputting an ultrasonic signal corrected in the element connected to the transmission channel based on the corrected transmission signal.

The obtaining of the post-correction parameter may include obtaining a first post-correction sub-parameter for correcting an acoustic characteristics deviation due to unique characteristics of at least one of a signal transmission/reception line within the main body, a signal transmission/reception line within the probe, or a signal transmission/reception line within a cable connecting the main body and the probe.

The obtaining of the post-correction parameter may include obtaining a second post-correction sub-parameter for correcting an acoustic characteristics deviation due to a connection relationship between the signal transmission/reception line within the probe and the signal transmission/reception line within the cable or a connection relationship between the signal transmission/reception line within the main body and the signal transmission/reception line within the cable.

The determining of the transmission parameter may include determining at least one of a depth, frequency, or waveform based on at least one of the object or the diagnosis subject.

An ultrasonic imaging apparatus according to an embodiment may comprises a main body comprising: a transmission module comprising a transmission channel configured to generate a transmission signal based on a synchronization signal, a main body communication module configured to perform communication with an external device including a probe, and a processor electrically connected to the transmission module and the main body communication module; and a probe comprising a plurality of elements configured to generate an ultrasonic signal based on the transmission signal, wherein the processor may be configured to obtain a pre-correction parameter about the probe based on connection of the main body and the probe, determine a transmission parameter based on at least one of an object or a diagnostic subject, correct at least one of a preset transmission delay time or a preset transmission intensity corresponding to the transmission parameter based on the pre-correction parameter, and control the transmission channel to generate a transmission signal based on at least one of the corrected transmission delay time or the corrected transmission intensity.

The pre-correction parameter may include a first pre-correction sub-parameter for correcting an acoustic characteristics deviation due to unique characteristics of at least one of an acoustic lens, a matching layer, each of a plurality of piezoelectric elements, or a sound-absorbing layer, which constitute the probe.

The pre-correction parameter may include a second pre-correction sub-parameter for correcting an acoustic characteristics deviation due to unique characteristics of an electrode unit configured to transmit the transmission signal to the plurality of piezoelectric elements.

The pre-correction parameter may include a third pre-correction sub-parameter for correcting an acoustic characteristics deviation due to unique characteristics of a type of the probe, and the third pre-correction sub-parameter may be a sub-parameter obtained as input data a plurality of acoustic characteristic pre-correction parameters corresponding to a plurality of respective probes of the same type as the probe using an artificial intelligence model.

The processor may be configured to control the main body communication module to receive the pre-correction parameter from the probe connected to the main body.

The processor may be configured to control the main body communication module to receive the pre-correction parameter from the external device, and the external device may include a server device or another main body.

The main body may further include a reception module including a reception channel configured to process an echo signal received by the probe, and the processor may be configured to control the reception channel to obtain processed data by processing the echo signal, obtain a post-correction parameter for correcting an acoustic characteristics deviation occurred in a process of transmitting and receiving a signal between the probe and the main body by using the processed data as input data using the artificial intelligence model, and control the transmission channel to correct the generated transmission signal based on the post-correction parameter.

The processor may be configured to correct the generated transmission signal based on the post-correction parameter and control the communication module to transmit the corrected transmission signal to the probe.

The post-correction parameter may include a first post-correction sub-parameter for correcting an acoustic characteristics deviation due to unique characteristics of at least one of a signal transmission/reception line within the main body, a signal transmission/reception line within the probe, or a signal transmission/reception line within a cable connecting the main body and the probe.

The post-correction parameter may include a second post-correction sub-parameter for correcting an acoustic characteristics deviation due to a connection relationship between the signal transmission/reception line within the main body and the signal transmission/reception line within the cable or a connection relationship between the signal transmission/reception line within the probe and the signal transmission/reception line within the cable.

The transmission parameter may include at least one of a depth, frequency or waveform.

As is apparent from the above, according to an aspect of the disclosure, an image of improved quality can be obtained by correcting a minute acoustic characteristics deviation.

According to an aspect of the disclosure, user experience can be improved.

However, effects that can be achieved by the ultrasonic imaging apparatus and the acoustic deviation correction method thereof according the disclosure are not limited to the effects mentioned above, and other effects not mentioned will be clearly understood by those skilled in the art to which the present disclosure belongs from the above description.

The disclosed embodiments may be implemented in the form of a recording medium storing instructions executable by a computer. The instructions may be stored in the form of program code, and when executed by a processor, a program module may be created to perform the operations of the disclosed embodiments. The recording medium may be implemented as a computer-readable recording medium.

The computer-readable recording medium includes any type of recording medium in which instructions readable by the computer are stored. For example, the recording medium may include a read only memory (ROM), a random access memory (RAM), a magnetic tape, a magnetic disk, a flash memory, an optical data storage device, and the like.

In addition, the computer-readable recording medium may be provided in the form of a non-transitory storage medium. Herein, the ‘non-transitory storage medium’ simply means that it is a tangible device and does not contain signals (e.g. electromagnetic waves), and this term does not distinguish between cases where data is semi-permanently stored in a storage medium and cases where data is stored temporarily. For example, the ‘non-transitory storage medium’ may include a buffer where data is temporarily stored.

According to an embodiment, methods according to various embodiments disclosed in this document may be provided and included in a computer program product. The computer program product is a commodity and may be traded between sellers and buyers. The computer program product may be distributed in the form of a machine-readable recording medium (e.g., compact disc read only memory (CD-ROM)), or may be distributed (e.g., downloaded or uploaded) online, through an application store (e.g., Play Store™) or directly between two user devices (e.g., smartphones). In the case of online distribution, at least a portion of the computer program product (e.g., a downloadable app) may be at least temporarily stored or created temporarily in the machine-readable recording medium, such as the memory of a manufacturer server, an application store server, or a relay server.

The foregoing has illustrated and described specific embodiments. However, it should be understood by those of skilled in the art that the disclosure is not limited to the above-described embodiments, and various changes and modifications may be made without departing from the technical idea of the disclosure described in the following claims.

What is claimed is:

1. An acoustic characteristics correction method of an ultrasonic imaging apparatus, which comprises a main body comprising a transmission channel configured to generate a transmission signal based on a synchronization signal, and a probe comprising a plurality of elements configured to generate an ultrasonic signal based on the transmission signal, comprising:
obtaining a pre-correction parameter about the probe based on connection of the main body and the probe;
determining a transmission parameter based on at least one of an object or a diagnostic subject;
correcting at least one of a preset transmission delay time or a preset transmission intensity corresponding to the transmission parameter based on the pre-correction parameter;
controlling the transmission channel to generate a transmission signal based on at least one of the corrected transmission delay time or the corrected transmission intensity; and
generating and outputting an ultrasonic signal in the element connected to the transmission channel based on the generated transmission signal.

2. The acoustic characteristics correction method according to claim 1, wherein
the obtaining of the pre-correction parameter comprises obtaining a first pre-correction sub-parameter for correcting an acoustic characteristics deviation due to unique characteristics of at least one of an acoustic lens, a plurality of piezoelectric elements, a matching layer, or a sound-absorbing layer, which constitute the probe.

3. The acoustic characteristics correction method according to claim 1, wherein
the obtaining of the pre-correction parameter comprises obtaining a second pre-correction sub-parameter for correcting an acoustic characteristics deviation due to unique characteristics of an electrode unit configured to transmit the transmission signal to a plurality of piezoelectric elements.

4. The acoustic characteristics correction method according to claim 1, wherein
the obtaining of the pre-correction parameter comprises obtaining a third pre-correction sub-parameter for correcting an acoustic characteristics deviation due to unique characteristics of a type of the probe, and
the third pre-correction sub-parameter is a sub-parameter obtained as input data a plurality of acoustic characteristic pre-correction parameters corresponding to a plurality of respective probes of the same type as the probe using an artificial intelligence model.

5. The acoustic characteristics correction method according to claim 1, wherein
the obtaining of the pre-correction parameter comprises controlling a main body communication module and a probe communication module to receive the pre-correction parameter from the probe connected to the main body.

6. The acoustic characteristics correction method according to claim 1, wherein
the ultrasonic imaging apparatus further comprises a reception channel configured to receive and process echo signals received by the plurality of elements, and the acoustic characteristics correction method comprises:
receiving an echo signal reflected from the object;
controlling the reception channel to obtain processed data by processing the echo signal;
obtaining a post-correction parameter for correcting an acoustic characteristics deviation occurred in a process of transmitting and receiving a signal between the probe and the main body by using the processed data as input data using the artificial intelligence model;
correcting the generated transmission signal based on the post-correction parameter; and
generating and outputting an ultrasonic signal corrected in the element connected to the transmission channel based on the corrected transmission signal.

7. The acoustic characteristics correction method according to claim 6, wherein
the obtaining of the post-correction parameter comprises obtaining a first post-correction sub-parameter for correcting an acoustic characteristics deviation due to unique characteristics of at least one of a signal transmission/reception line within the main body, a signal transmission/reception line within the probe, or a signal transmission/reception line within a cable connecting the main body and the probe.

8. The acoustic characteristics correction method according to claim 6, wherein
the obtaining of the post-correction parameter comprises obtaining a second post-correction sub-parameter for correcting an acoustic characteristics deviation due to a connection relationship between a signal transmission/reception line within the probe and a signal transmission/reception line within a cable connecting the main body and the probe or a connection relationship between a signal transmission/reception line within the main body and the signal transmission/reception line within the cable.

9. The acoustic characteristics correction method according to claim 1, wherein
the determining of the transmission parameter comprises determining at least one of a depth, frequency, or waveform based on at least one of the object or the diagnosis subject.

10. An ultrasonic imaging apparatus comprising:
a main body comprising:
a transmission module comprising a transmission channel configured to generate a transmission signal based on a synchronization signal,
a main body communication module configured to perform communication with an external device including a probe, and
a processor electrically connected to the transmission module and the main body communication module; and
a probe comprising a plurality of elements configured to generate an ultrasonic signal based on the transmission signal,
wherein the processor is configured to obtain a pre-correction parameter about the probe based on connection of the main body and the probe,
determine a transmission parameter based on at least one of an object or a diagnostic subject,
correct at least one of a preset transmission delay time or a preset transmission intensity corresponding to the transmission parameter based on the pre-correction parameter, and
control the transmission channel to generate a transmission signal based on at least one of the corrected transmission delay time or the corrected transmission intensity.

11. The ultrasonic imaging apparatus according to claim 10, wherein
the pre-correction parameter comprises a first pre-correction sub-parameter for correcting an acoustic characteristics deviation due to unique characteristics of at least one of an acoustic lens, a matching layer, a plurality of piezoelectric elements, or a sound-absorbing layer, which constitute the probe.

12. The ultrasonic imaging apparatus according to claim 10, wherein
the pre-correction parameter comprises a second pre-correction sub-parameter for correcting an acoustic characteristics deviation due to unique characteristics of an electrode unit configured to transmit the transmission signal to a plurality of piezoelectric elements.

13. The ultrasonic imaging apparatus according to claim 10, wherein
the pre-correction parameter comprises a third pre-correction sub-parameter for correcting an acoustic characteristics deviation due to unique characteristics of a type of the probe, and
the third pre-correction sub-parameter may be a sub-parameter obtained as input data a plurality of acoustic characteristic pre-correction parameters corresponding to a plurality of respective probes of the same type as the probe using an artificial intelligence model.

14. The ultrasonic imaging apparatus according to claim 10, wherein
the processor is configured to control the main body communication module to receive the pre-correction parameter from the probe connected to the main body.

15. The ultrasonic imaging apparatus according to claim 10, wherein
the processor is configured to control the main body communication module to receive the pre-correction parameter from the external device, and
the external device comprises a server device or another main body.

16. The ultrasonic imaging apparatus according to claim 10, wherein
the main body further comprises a reception module comprising a reception channel configured to process an echo signal received by the probe,
and the processor is configured to
control the reception channel to obtain processed data by processing the echo signal,
obtain a post-correction parameter for correcting an acoustic characteristics deviation occurred in a process of transmitting and receiving a signal between the probe and the main body by using the processed data as input data using the artificial intelligence model, and
control the transmission channel to correct the generated transmission signal based on the post-correction parameter.

17. The ultrasonic imaging apparatus according to claim 16, wherein
the processor is configured to
correct the generated transmission signal based on the post-correction parameter, and
control the main body communication module to transmit the corrected transmission signal to the probe.

18. The ultrasonic imaging apparatus according to claim 16, wherein
the post-correction parameter comprises a first post-correction sub-parameter for correcting an acoustic characteristics deviation due to unique characteristics of at least one of a signal transmission/reception line within the main body, a signal transmission/reception line within the probe, or a signal transmission/reception line within a cable connecting the main body and the probe.

19. The ultrasonic imaging apparatus according to claim 16, wherein
the post-correction parameter comprises a second post-correction sub-parameter for correcting an acoustic characteristics deviation due to a connection relationship between a signal transmission/reception line within the main body and a signal transmission/reception line within a cable or a connection relationship between a signal transmission/reception line within the probe and the signal transmission/reception line within the cable.

20. The ultrasonic imaging apparatus according to claim 10, wherein
the transmission parameter comprises at least one of a depth, frequency or waveform.
